(19) United States
(12) Patent Application Publication
(10) Pub. No.: US 2022/0296895 A1
(43) Pub. Date: Sep. 22, 2022

(54) Optimizing an Individual's Wellness Therapy Using a Non-Invasive Brain Measurement System

(71) Applicant: HI LLC, Los Angeles, CA (US)

(72) Inventors: Bryan Johnson, Culver City, CA (US); Ryan Field, Culver City, CA (US)

(21) Appl. No.: 17/572,281

(22) Filed: Jan. 10, 2022

Related U.S. Application Data
(60) Provisional application No. 63/164,343, filed on Mar. 22, 2021.
(60) Provisional application No. 63/188,783, filed on May 14, 2021.

(51) Int. Cl. A61N 1/36; A61N 1/04; G01R 33/02; G16H 40/63

(52) U.S. Cl. CPC A61N 1/36031; A61N 1/0456; G01R 33/0206; G16H 40/63

(57) ABSTRACT

An illustrative system includes a brain interface system configured to be worn by a user and to output brain measurement data representative of brain activity of the user while the user concurrently receives a wellness therapy and a computing device configured to obtain the brain measurement data, and modify, based on the brain measurement data, an attribute of the wellness therapy.

### RELATED APPLICATIONS

The present application claims priority under 35 U.S.C. § 119(e) to U.S. Provisional Patent Application Nos. 63/164,343, filed Mar. 22, 2021, and 63/188,783, filed May 14, 2021. These applications are incorporated herein by reference in their respective entireties.

### BACKGROUND INFORMATION

A user may receive wellness therapy (e.g., stimulation applied by a non-invasive stimulation device, prescription medication, etc.) to treat a condition of the user. Evaluation of an effectiveness of such treatment is often subjective, and therefore prone to inaccuracies. As such, real-time insight into how a wellness therapy affects a user while the user receives the wellness therapy may result in an improved experience and outcome for the user.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary configuration that includes a brain interface system and a computing device.

FIGS. 2-4, 5A and 5B show various optical measurement systems that may implement the brain interface system shown in FIG. 1.

FIGS. 6-7 show various multimodal measurement systems that may implement the brain interface system shown in FIG. 1.

FIG. 8 shows an exemplary magnetic field measurement system that may implement the brain interface system shown in FIG. 1.

FIG. 9 shows an exemplary configuration in which a stimulation system presents a stimulation therapy to a user and outputs stimulation program data associated with the stimulation therapy.

FIG. 10 shows an exemplary configuration in which a pharmaceutical therapy is presented to a user and the system outputs pharmaceutical control data associated with the pharmaceutical therapy.

FIG. 11 shows an exemplary configuration in which a sensor outputs sensor data associated with the user.

FIG. 12 shows an illustrative configuration in which a computing device is configured to implement a machine learning model to perform one or more operations based on the brain measurement data output by brain interface system.

FIGS. 13-14 illustrate exemplary methods.

FIG. 15 illustrates an exemplary computing device.

### DETAILED DESCRIPTION

Optimizing an individual's wellness therapy using a non-invasive brain measurement system is described herein. For example, an illustrative non-invasive brain measurement system may include a brain interface system and a computing device. The brain interface system may be configured to be worn by a user and to output brain measurement data representative of brain activity of the user while the user concurrently receives a wellness therapy. The computing device may be configured to obtain the brain measurement data and modify, based on the brain measurement data, an attribute of the wellness therapy.

By tracking the user's brain activity while the user concurrently receives the wellness therapy, various benefits may be realized for the user and/or others. For example, the tracked brain activity may be used to determine an efficacy of the wellness therapy. Additionally or alternatively, based on the user's brain activity, the wellness therapy may be adjusted (e.g., in substantially real time as the user receives the wellness therapy) to optimize and individualize the wellness therapy to treat the condition for which the user is receiving the wellness therapy. These and other benefits are described more fully herein.

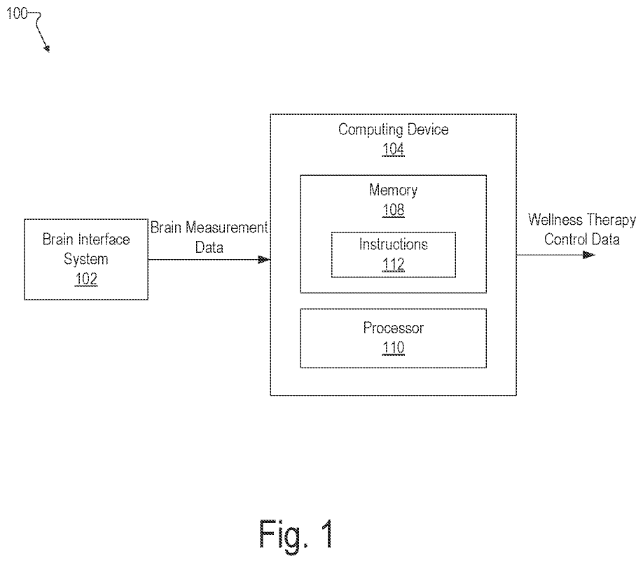

FIG. 1 shows an exemplary non-invasive brain measurement system 100 that includes a brain interface system 102 and a computing device 104.

Brain interface system 102 may be configured to be worn by a user and to output brain measurement data representative of brain activity of the user while the brain interface system 102 is being worn by the user. As described herein, the brain measurement data may include any data output by any of the implementations of brain interface system 102 described herein. For example, the brain measurement data may include or be based on optical-based, electrical-based, and/or magnetic field-based measurements of activity within the brain, as described herein.

Computing device 104 may be configured to obtain (e.g., receive or otherwise access) the brain measurement data. This may be performed in any suitable manner. For example, computing device 104 may receive the brain measurement data from brain interface system 102 by way a wired and/or wireless (e.g., Bluetooth, WiFi, etc.) connection.

Computing device 104 may be further configured to modify, based on the brain measurement data, an attribute of a wellness therapy that the user is concurrently receiving. To this end, computing device 104 may generate wellness therapy control data based on the brain measurement data. Examples of wellness therapy control data that computing device 104 may determine based on brain measurement data are described herein.

In some examples, brain interface system 102 may output the brain measurement data in substantially real time (e.g., concurrently) while the user receives the wellness therapy. Furthermore, computing device 104 may obtain the brain measurement data and modify the attribute of the wellness therapy in substantially real time (e.g., concurrently) while the user receives the wellness therapy. As used herein, “real time” and “substantially real time” and “concurrently” will be understood to relate to data processing and/or other actions that are performed immediately, as well as conditions and/or circumstances that are accounted for as they exist in the moment, or at the same time, when the processing or other actions are performed. For example, a real-time operation may refer to an operation that is performed immediately and without undue delay, even if it is not possible for there to be absolutely zero delay. Similarly, real-time data, real-time representations, real-time conditions, at the same time conditions, and so forth, will be understood to refer to data, representations, and conditions that relate to a present moment in time or a moment in time when decisions are being made and operations are being performed (e.g., even if after a short delay), such that the data, representations, conditions, and so forth are temporally relevant to the decisions being made and/or the operations being performed.

Computing device 104 may be implemented by one or more computing or processing devices, such as one or more personal computers, mobile devices (e.g., a mobile phone, a tablet computer, etc.), servers, and/or any other type of computing device as may serve a particular implementation. In some examples, computing device 104 may be included in brain interface system 102. Additionally or alternatively, computing device 104 may be separate from (i.e., remote from and communicatively coupled to) brain interface system 102.

As shown, computing device 104 may include memory 108 and a processor 110. Computing device 104 may include additional or alternative components as may serve a particular implementation. Each component may be implemented by any suitable combination of hardware and/or software.

Memory 108 may maintain (e.g., store) executable data used by processor 110 to perform one or more of the operations described herein as being performed by computing device 104. For example, memory 108 may store instructions 112 that may be executed by processor 110 to generate wellness therapy control data and/or perform one or more operations based on the wellness therapy control data. Instructions 112 may be implemented by any suitable application, program, software, code, and/or other executable data instance. Memory 108 may also maintain any data received, generated, managed, used, and/or transmitted by processor 110.

Processor 110 may be configured to perform (e.g., execute instructions 112 stored in memory 108 to perform) various operations described herein as being performed by computing device 104. Examples of such operations are described herein.

Brain interface system 102 may be implemented by any suitable wearable non-invasive brain interface system as may serve a particular implementation. For example, brain interface system 102 may be implemented by a wearable optical measurement system configured to perform optical-based brain data acquisition operations, such as any of the wearable optical measurement systems described in U.S. patent application Ser. No. 17/176,315, filed Feb. 16, 2021 and published as US2021/0259638A1; U.S. patent application Ser. No. 17/176,309, filed Feb. 16, 2021 and published as US2021/0259614A1; U.S. patent application Ser. No. 17/176,460, filed Feb. 16, 2021, issued as U.S. Pat. No. 11,096,620; U.S. patent application Ser. No. 17/176,470, filed Feb. 16, 2021 and published as US2021/0259619A1; U.S. patent application Ser. No. 17/176,487, filed Feb. 16, 2021 and published as US2021/0259632A1; U.S. patent application Ser. No. 17/176,539, filed Feb. 16, 2021 and published as US2021/0259620A1; U.S. patent application Ser. No. 17/176,560, filed Feb. 16, 2021 and published as US2021/0259597A1; U.S. patent application Ser. No. 17/176,466, filed Feb. 16, 2021 and published as US2021/0263320A1; and Han Y. Ban, et al., “Kernel Flow: A High Channel Count Scalable TD-fNIRS System,” SPIE Photonics West Conference (Mar. 6, 2021), which applications and publications are incorporated herein by reference in their entirety.

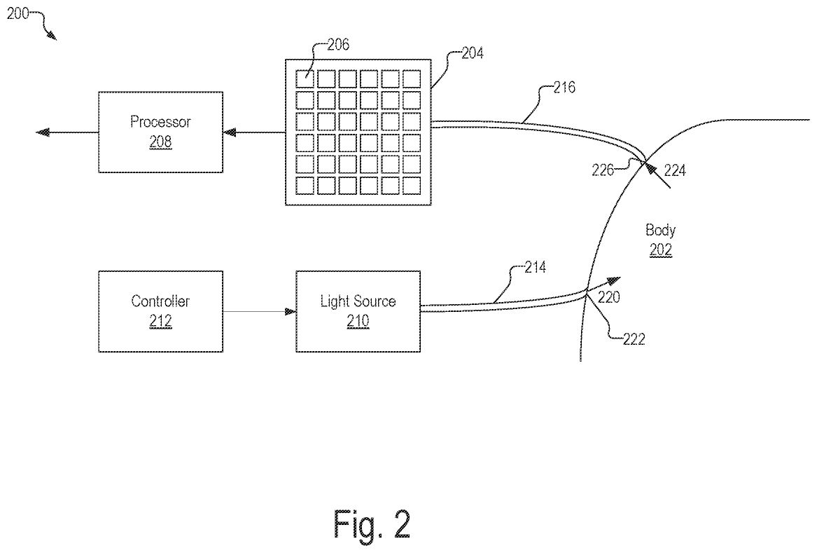

To illustrate, FIGS. 2-4, 5A, and 5B, show various optical measurement systems and related components that may implement brain interface system 102. The optical measurement systems described herein are merely illustrative of the many different optical-based brain interface systems that may be used in accordance with the systems and methods described herein.

FIG. 2 shows an optical measurement system 200 that may be configured to perform an optical measurement operation with respect to a body 202 (e.g., the brain). Optical measurement system 200 may, in some examples, be portable and/or wearable by a user.

In some examples, optical measurement operations performed by optical measurement system 200 are associated with a time domain-based optical measurement technique. Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain digital optical tomography (TD-DOT).

Optical measurement system 200 (e.g., an optical measurement system that is implemented by a wearable device or other configuration, and that employs a time domain-based (e.g., TD-NIRS) measurement technique) may detect blood oxygenation levels and/or blood volume levels by measuring the change in shape of laser pulses after they have passed through target tissue, e.g., brain, muscle, finger, etc. As used herein, a shape of laser pulses refers to a temporal shape, as represented for example by a histogram generated by a time-to-digital converter (TDC) coupled to an output of a photodetector, as will be described more fully below.

As shown, optical measurement system 200 includes a detector 204 that includes a plurality of individual photodetectors (e.g., photodetector 206), a processor 208 coupled to detector 204, a light source 210, a controller 212, and optical conduits 214 and 216 (e.g., light pipes). However, one or more of these components may not, in certain embodiments, be considered to be a part of optical measurement system 200. For example, in implementations where optical measurement system 200 is wearable by a user, processor 208 and/or controller 212 may in some embodiments be separate from optical measurement system 200 and not configured to be worn by the user.

Detector 204 may include any number of photodetectors 206 as may serve a particular implementation, such as 2ⁿ photodetectors (e.g., 256, 512, . . . , 26384, etc.), where n is an integer greater than or equal to one (e.g., 4, 5, 8, 20, 21, 24, etc.). Photodetectors 206 may be arranged in any suitable manner.

Photodetectors 206 may each be implemented by any suitable circuit configured to detect individual photons of light incident upon photodetectors 206. For example, each photodetector 206 may be implemented by a single photon avalanche diode (SPAD) circuit and/or other circuitry as may serve a particular implementation. The SPAD circuit may be gated in any suitable manner or be configured to operate in a free running mode with passive quenching. For example, photodetectors 206 may be configured to operate in a free-running mode such that photodetectors 206 are not actively armed and disarmed (e.g., at the end of each predetermined gated time window). In contrast, while operating in the free-running mode, photodetectors 206 may be configured to reset within a configurable time period after an occurrence of a photon detection event (i.e., after photodetector 206 detects a photon) and immediately begin detecting new photons. However, only photons detected within a desired time window (e.g., during each gated time window) may be included in the histogram that represents a light pulse response of the target (e.g., a temporal point spread function (TPSF)). The terms histogram and TPSF are used interchangeably herein to refer to a light pulse response of a target.

Processor 208 may be implemented by one or more physical processing (e.g., computing) devices. In some examples, processor 208 may execute instructions (e.g., software) configured to perform one or more of the operations described herein.

Light source 210 may be implemented by any suitable component configured to generate and emit light. For example, light source 210 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 210 is high coherence light (e.g., light that has a coherence length of at least 5 centimeters) at a predetermined center wavelength.

Light source 210 is controlled by controller 212, which may be implemented by any suitable computing device (e.g., processor 208), integrated circuit, and/or combination of hardware and/or software as may serve a particular implementation. In some examples, controller 212 is configured to control light source 210 by turning light source 210 on and off and/or setting an intensity of light generated by light source 210. Controller 212 may be manually operated by a user, or may be programmed to control light source 210 automatically.

Light emitted by light source 210 may travel via an optical conduit 214 (e.g., a light pipe, a single-mode optical fiber, and/or or a multi-mode optical fiber) to body 202 of a subject. Body 202 may include any suitable turbid medium. For example, in some implementations, body 202 is a brain or any other body part of a human or other animal. Alternatively, body 202 may be a non-living object. For illustrative purposes, it will be assumed in the examples provided herein that body 202 is a human brain.

As indicated by arrow 220, the light emitted by light source 210 enters body 202 at a first location 222 on body 202. Accordingly, a distal end of optical conduit 214 may be positioned at (e.g., right above, in physical contact with, or physically attached to) first location 222 (e.g., to a scalp of the subject). In some examples, the light may emerge from optical conduit 214 and spread out to a certain spot size on body 202 to fall under a predetermined safety limit. At least a portion of the light indicated by arrow 220 may be scattered within body 202.

As used herein, “distal” means nearer, along the optical path of the light emitted by light source 210 or the light received by detector 204, to the target (e.g., within body 202) than to light source 210 or detector 204. Thus, the distal end of optical conduit 214 is nearer to body 202 than to light source 210, and the distal end of optical conduit 216 is nearer to body 202 than to detector 204. Additionally, as used herein, “proximal” means nearer, along the optical path of the light emitted by light source 210 or the light received by detector 204, to light source 210 or detector 204 than to body 202. Thus, the proximal end of optical conduit 214 is nearer to light source 210 than to body 202, and the proximal end of optical conduit 216 is nearer to detector 204 than to body 202.

As shown, the distal end of optical conduit 216 (e.g., a light pipe, a light guide, a waveguide, a single-mode optical fiber, and/or a multi-mode optical fiber) is positioned at (e.g., right above, in physical contact with, or physically attached to) output location 226 on body 202. In this manner, optical conduit 216 may collect at least a portion of the scattered light (indicated as light 224) as it exits body 202 at location 226 and carry light 224 to detector 204. Light 224 may pass through one or more lenses and/or other optical elements (not shown) that direct light 224 onto each of the photodetectors 206 included in detector 204. In cases where optical conduit 216 is implemented by a light guide, the light guide may be spring loaded and/or have a cantilever mechanism to allow for conformably pressing the light guide firmly against body 202.

Photodetectors 206 may be connected in parallel in detector 204. An output of each of photodetectors 206 may be accumulated to generate an accumulated output of detector 204. Processor 208 may receive the accumulated output and determine, based on the accumulated output, a temporal distribution of photons detected by photodetectors 206. Processor 208 may then generate, based on the temporal distribution, a histogram representing a light pulse response of a target (e.g., brain tissue, blood flow, etc.) in body 202. Such a histogram is illustrative of the various types of brain activity measurements that may be performed by brain interface system 102.

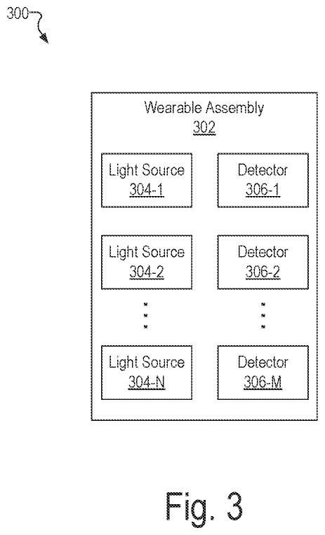

FIG. 3 shows an exemplary optical measurement system 300 in accordance with the principles described herein. Optical measurement system 300 may be an implementation of optical measurement system 200 and, as shown, includes a wearable assembly 302, which includes N light sources 304 (e.g., light sources 304-1 through 304-N) and M detectors 306 (e.g., detectors 306-1 through 306-M). Optical measurement system 300 may include any of the other components of optical measurement system 200 as may serve a particular implementation. N and M may each be any suitable value (i.e., there may be any number of light sources 304 and detectors 306 included in optical measurement system 300 as may serve a particular implementation).

Light sources 304 are each configured to emit light (e.g., a sequence of light pulses) and may be implemented by any of the light sources described herein. Detectors 306 may each be configured to detect arrival times for photons of the light emitted by one or more light sources 304 after the light is scattered by the target. For example, a detector 306 may include a photodetector configured to generate a photodetector output pulse in response to detecting a photon of the light and a time-to-digital converter (TDC) configured to record a timestamp symbol in response to an occurrence of the photodetector output pulse, the timestamp symbol representative of an arrival time for the photon (i.e., when the photon is detected by the photodetector).

Wearable assembly 302 may be implemented by any of the wearable devices, modular assemblies, and/or wearable units described herein. For example, wearable assembly 302 may be implemented by a wearable device (e.g., headgear) configured to be worn on a user's head. Wearable assembly 302 may additionally or alternatively be configured to be worn on any other part of a user's body.

Optical measurement system 300 may be modular in that one or more components of optical measurement system 300 may be removed, changed out, or otherwise modified as may serve a particular implementation. As such, optical measurement system 300 may be configured to conform to three-dimensional surface geometries, such as a user's head. Exemplary modular optical measurement systems comprising a plurality of wearable modules are described in more detail in one or more of the patent applications incorporated herein by reference.

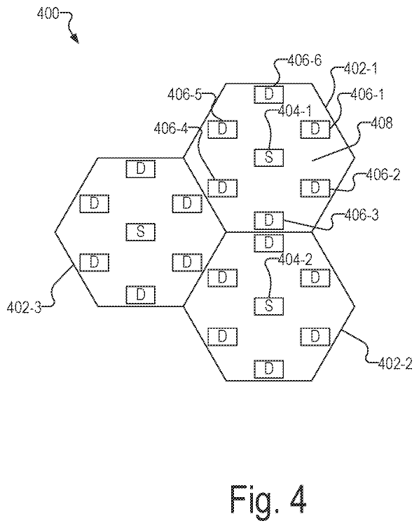

FIG. 4 shows an illustrative modular assembly 400 that may implement optical measurement system 300. Modular assembly 400 is illustrative of the many different implementations of optical measurement system 300 that may be realized in accordance with the principles described herein.

As shown, modular assembly 400 includes a plurality of modules 402 (e.g., modules 402-1 through 402-3) physically distinct one from another. While three modules 402 are shown to be included in modular assembly 400, in alternative configurations, any number of modules 402 (e.g., a single module up to sixteen or more modules) may be included in modular assembly 400.

Each module 402 includes a light source (e.g., light source 404-1 of module 402-1 and light source 404-2 of module 402-2) and a plurality of detectors (e.g., detectors 406-1 through 406-6 of module 402-1). In the particular implementation shown in FIG. 4, each module 402 includes a single light source and six detectors. Each light source is labeled “S” and each detector is labeled “D”.

Each light source depicted in FIG. 4 may be implemented by one or more light sources similar to light source 210 and may be configured to emit light directed at a target (e.g., the brain).

Each light source depicted in FIG. 4 may be located at a center region of a surface of the light source's corresponding module. For example, light source 404-1 is located at a center region of a surface 408 of module 402-1. In alternative implementations, a light source of a module may be located away from a center region of the module.

Each detector depicted in FIG. 4 may implement or be similar to detector 204 and may include a plurality of photodetectors (e.g., SPADs) as well as other circuitry (e.g., TDCs), and may be configured to detect arrival times for photons of the light emitted by one or more light sources after the light is scattered by the target.

The detectors of a module may be distributed around the light source of the module. For example, detectors 406 of module 402-1 are distributed around light source 404-1 on surface 408 of module 402-1. In this configuration, detectors 406 may be configured to detect photon arrival times for photons included in light pulses emitted by light source 404-1. In some examples, one or more detectors 406 may be close enough to other light sources to detect photon arrival times for photons included in light pulses emitted by the other light sources. For example, because detector 406-3 is adjacent to module 402-2, detector 406-3 may be configured to detect photon arrival times for photons included in light pulses emitted by light source 404-2 (in addition to detecting photon arrival times for photons included in light pulses emitted by light source 404-1).

In some examples, the detectors of a module may all be equidistant from the light source of the same module. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light source, e.g., light emitter, and the detector allows subsequent processing of the detected signals to infer spatial (e.g., depth localization, inverse modeling) information about the detected signals. Detectors of a module may be alternatively disposed on the module as may serve a particular implementation.

In some examples, modular assembly 400 can conform to a three-dimensional (3D) surface of the human subject's head, maintain tight contact of the detectors with the human subject's head to prevent detection of ambient light, and maintain uniform and fixed spacing between light sources and detectors. The wearable module assemblies may also accommodate a large variety of head sizes, from a young child's head size to an adult head size, and may accommodate a variety of head shapes and underlying cortical morphologies through the conformability and scalability of the wearable module assemblies. These exemplary modular assemblies and systems are described in more detail in U.S. patent application Ser. Nos. 17/176,470; 17/176,487; 17/176,539; 17/176,560; 17/176,460; and Ser. No. 17/176,466, which applications have been previously incorporated herein by reference in their respective entireties.

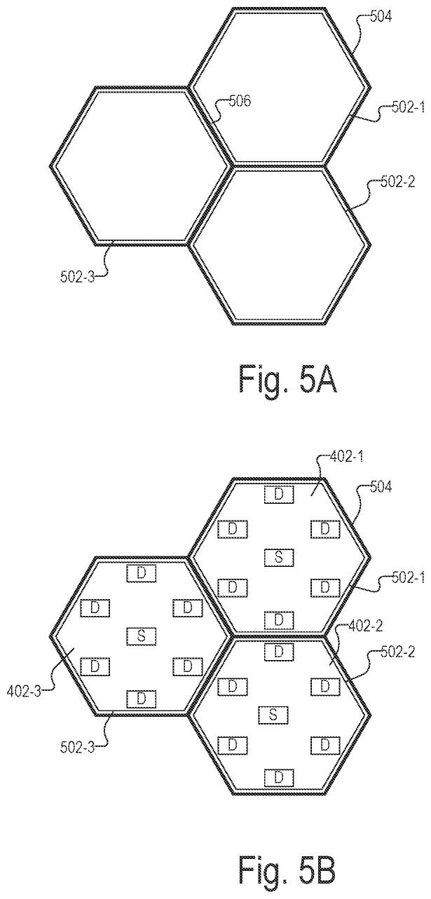

In FIG. 4, modules 402 are shown to be adjacent to and touching one another. Modules 402 may alternatively be spaced apart from one another. For example, FIGS. 5A-5B show an exemplary implementation of modular assembly 400 in which modules 402 are configured to be inserted into individual slots 502 (e.g., slots 502-1 through 502-3, also referred to as cutouts) of a wearable assembly 504. In particular, FIG. 5A shows the individual slots 502 of the wearable assembly 504 before modules 402 have been inserted into respective slots 502, and FIG. 5B shows wearable assembly 504 with individual modules 402 inserted into respective individual slots 502.

Wearable assembly 504 may implement wearable assembly 302 and may be configured as headgear and/or any other type of device configured to be worn by a user.

As shown in FIG. 5A, each slot 502 is surrounded by a wall (e.g., wall 506) such that when modules 402 are inserted into their respective individual slots 502, the walls physically separate modules 402 one from another. In alternative embodiments, a module (e.g., module 402-1) may be in at least partial physical contact with a neighboring module (e.g., module 402-2).

Each of the modules described herein may be inserted into appropriately shaped slots or cutouts of a wearable assembly, as described in connection with FIGS. 5A-5B. However, for ease of explanation, such wearable assemblies are not shown in the figures.

As shown in FIGS. 4 and 5B, modules 402 may have a hexagonal shape. Modules 402 may alternatively have any other suitable geometry (e.g., in the shape of a pentagon, octagon, square, rectangular, circular, triangular, free-form, etc.).

As another example, brain interface system 102 may be implemented by a wearable multimodal measurement system configured to perform both optical-based brain data acquisition operations and electrical-based brain data acquisition operations, such as any of the wearable multimodal measurement systems described in U.S. patent application Ser. Nos. 17/176,315 and 17/176,309, which applications have been previously incorporated herein by reference in their respective entireties.

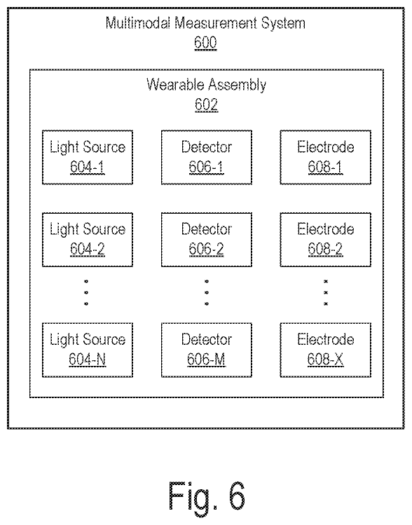

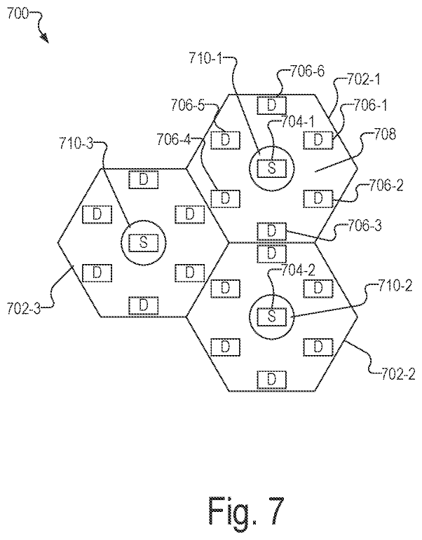

To illustrate, FIGS. 6-7 show various multimodal measurement systems that may implement brain interface system 102. The multimodal measurement systems described herein are merely illustrative of the many different multimodal-based brain interface systems that may be used in accordance with the systems and methods described herein.

FIG. 6 shows an exemplary multimodal measurement system 600 in accordance with the principles described herein. Multimodal measurement system 600 may at least partially implement optical measurement system 200 and, as shown, includes a wearable assembly 602 (which is similar to wearable assembly 302), which includes N light sources 604 (e.g., light sources 604-1 through 604-N, which are similar to light sources 304), M detectors 606 (e.g., detectors 606-1 through 606-M, which are similar to detectors 306), and X electrodes (e.g., electrodes 608-1 through 608-X). Multimodal measurement system 600 may include any of the other components of optical measurement system 200 as may serve a particular implementation. N, M, and X may each be any suitable value (i.e., there may be any number of light sources 604, any number of detectors 606, and any number of electrodes 608 included in multimodal measurement system 600 as may serve a particular implementation).

Electrodes 608 may be configured to detect electrical activity within a target (e.g., the brain). Such electrical activity may include electroencephalogram (EEG) activity and/or any other suitable type of electrical activity as may serve a particular implementation. In some examples, electrodes 608 are all conductively coupled to one another to create a single channel that may be used to detect electrical activity. Alternatively, at least one electrode included in electrodes 608 is conductively isolated from a remaining number of electrodes included in electrodes 608 to create at least two channels that may be used to detect electrical activity.

FIG. 7 shows an illustrative modular assembly 700 that may implement multimodal measurement system 600. As shown, modular assembly 700 includes a plurality of modules 702 (e.g., modules 702-1 through 702-3). While three modules 702 are shown to be included in modular assembly 700, in alternative configurations, any number of modules 702 (e.g., a single module up to sixteen or more modules) may be included in modular assembly 700. Moreover, while each module 702 has a hexagonal shape, modules 702 may alternatively have any other suitable geometry (e.g., in the shape of a pentagon, octagon, square, rectangular, circular, triangular, free-form, etc.).

Each module 702 includes a light source (e.g., light source 704-1 of module 702-1 and light source 704-2 of module 702-2) and a plurality of detectors (e.g., detectors 706-1 through 706-6 of module 702-1). In the particular implementation shown in FIG. 7, each module 702 includes a single light source and six detectors. Alternatively, each module 702 may have any other number of light sources (e.g., two light sources) and any other number of detectors. The various components of modular assembly 700 shown in FIG. 7 are similar to those described in connection with FIG. 4.

As shown, modular assembly 700 further includes a plurality of electrodes 710 (e.g., electrodes 710-1 through 710-3), which may implement electrodes 608. Electrodes 710 may be located at any suitable location that allows electrodes 710 to be in physical contact with a surface (e.g., the scalp and/or skin) of a body of a user. For example, in modular assembly 700, each electrode 710 is on a module surface configured to face a surface of a user's body when modular assembly 700 is worn by the user. To illustrate, electrode 710-1 is on surface 708 of module 702-1. Moreover, in modular assembly 700, electrodes 710 are located in a center region of each module 702 and surround each module's light source 704. Alternative locations and configurations for electrodes 710 are possible.

As another example, brain interface system 102 may be implemented by a wearable magnetic field measurement system configured to perform magnetic field-based brain data acquisition operations, such as any of the magnetic field measurement systems described in U.S. patent application Ser. No. 16/862,879, filed Apr. 30, 2020 and published as US2020/0348368A1; U.S. Provisional Application No. 63/170,892, filed Apr. 5, 2021, U.S. Non-Provisional application Ser. No. 17/338,429, filed Jun. 3, 2021, and Ethan J. Pratt, et al., “Kernel Flux: A Whole-Head 432-Magnetometer Optically-Pumped Magnetoencephalography (OP-MEG) System for Brain Activity Imaging During Natural Human Experiences,” SPIE Photonics West Conference (Mar. 6, 2021), which applications and publications are incorporated herein by reference in their entirety. In some examples, any of the magnetic field measurement systems described herein may be used in a magnetically shielded environment which allows for natural user movement as described for example in U.S. Provisional Patent Application No. 63/076,015, filed Sep. 9, 2020, and U.S. Non-Provisional patent application Ser. No. 17/328,235, filed May 24, 2021 and published as US2021/0369166A1, which applications are incorporated herein by reference in their entirety.

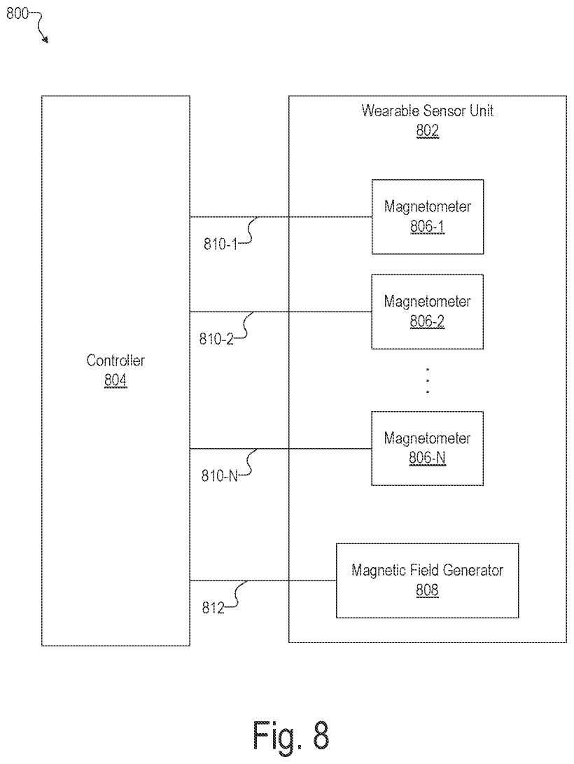

FIG. 8 shows an exemplary magnetic field measurement system 800 (“system 800”) that may implement brain interface system 102. As shown, system 800 includes a wearable sensor unit 802 and a controller 804. Wearable sensor unit 802 includes a plurality of magnetometers 806-1 through 806-N (collectively “magnetometers 806”, also referred to as optically pumped magnetometer (OPM) modular assemblies as described below) and a magnetic field generator 808. Wearable sensor unit 802 may include additional components (e.g., one or more magnetic field sensors, position sensors, orientation sensors, accelerometers, image recorders, detectors, etc.) as may serve a particular implementation. System 800 may be used in magnetoencephalography (MEG) and/or any other application that measures relatively weak magnetic fields.

Wearable sensor unit 802 is configured to be worn by a user (e.g., on a head of the user). In some examples, wearable sensor unit 802 is portable. In other words, wearable sensor unit 802 may be small and light enough to be easily carried by a user and/or worn by the user while the user moves around and/or otherwise performs daily activities, or may be worn in a magnetically shielded environment which allows for natural user movement as described more fully in U.S. Provisional Patent Application No. 63/076,015, and U.S. Non-Provisional patent application Ser. No. 17/328,235, filed May 24, 2021 and published as US2021/0369166A1, previously incorporated by reference.

Any suitable number of magnetometers 806 may be included in wearable sensor unit 802. For example, wearable sensor unit 802 may include an array of nine, sixteen, twenty-five, or any other suitable plurality of magnetometers 806 as may serve a particular implementation.

Magnetometers 806 may each be implemented by any suitable combination of components configured to be sensitive enough to detect a relatively weak magnetic field (e.g., magnetic fields that come from the brain). For example, each magnetometer may include a light source, a vapor cell such as an alkali metal vapor cell (the terms “cell”, “gas cell”, “vapor cell”, and “vapor gas cell” are used interchangeably herein), a heater for the vapor cell, and a photodetector (e.g., a signal photodiode). Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source may include two light sources: a pump light source and a probe light source.

Magnetic field generator 808 may be implemented by one or more components configured to generate one or more compensation magnetic fields that actively shield magnetometers 806 (including respective vapor cells) from ambient background magnetic fields (e.g., the Earth's magnetic field, magnetic fields generated by nearby magnetic objects such as passing vehicles, electrical devices and/or other field generators within an environment of magnetometers 806, and/or magnetic fields generated by other external sources). For example, magnetic field generator 808 may include one or more coils configured to generate compensation magnetic fields in the Z direction, X direction, and/or Y direction (all directions are with respect to one or more planes within which the magnetic field generator 808 is located). The compensation magnetic fields are configured to cancel out, or substantially reduce, ambient background magnetic fields in a magnetic field sensing region with minimal spatial variability.

Controller 804 is configured to interface with (e.g., control an operation of, receive signals from, etc.) magnetometers 806 and the magnetic field generator 808. Controller 804 may also interface with other components that may be included in wearable sensor unit 802.

In some examples, controller 804 is referred to herein as a “single” controller 804. This means that only one controller is used to interface with all of the components of wearable sensor unit 802. For example, controller 804 may be the only controller that interfaces with magnetometers 806 and magnetic field generator 808. It will be recognized, however, that any number of controllers may interface with components of magnetic field measurement system 800 as may suit a particular implementation.

As shown, controller 804 may be communicatively coupled to each of magnetometers 806 and magnetic field generator 808. For example, FIG. 8 shows that controller 804 is communicatively coupled to magnetometer 806-1 by way of communication link 810-1, to magnetometer 806-2 by way of communication link 810-2, to magnetometer 806-N by way of communication link 810-N, and to magnetic field generator 808 by way of communication link 812. In this configuration, controller 804 may interface with magnetometers 806 by way of communication links 810-1 through 810-N (collectively “communication links 810”) and with magnetic field generator 808 by way of communication link 812.

Communication links 810 and communication link 812 may be implemented by any suitable wired connection as may serve a particular implementation. For example, communication links 810 may be implemented by one or more twisted pair cables while communication link 812 may be implemented by one or more coaxial cables. Alternatively, communication links 810 and communication link 812 may both be implemented by one or more twisted pair cables. In some examples, the twisted pair cables may be unshielded.

Controller 804 may be implemented in any suitable manner. For example, controller 804 may be implemented by a field-programmable gate array (FPGA), an application specific integrated circuit (ASIC), a digital signal processor (DSP), a microcontroller, and/or other suitable circuit together with various control circuitry.

In some examples, controller 804 is implemented on one or more printed circuit boards (PCBs) included in a single housing. In cases where controller 804 is implemented on a PCB, the PCB may include various connection interfaces configured to facilitate communication links 810 and 812. For example, the PCB may include one or more twisted pair cable connection interfaces to which one or more twisted pair cables may be connected (e.g., plugged into) and/or one or more coaxial cable connection interfaces to which one or more coaxial cables may be connected (e.g., plugged into).

In some examples, controller 804 may be implemented by or within a computing device.

In some examples, a wearable magnetic field measurement system may include a plurality of optically pumped magnetometer (OPM) modular assemblies, which OPM modular assemblies are enclosed within a housing sized to fit into a headgear (e.g., brain interface system 102) for placement on a head of a user (e.g., human subject). The OPM modular assembly is designed to enclose the elements of the OPM optics, vapor cell, and detectors in a compact arrangement that can be positioned close to the head of the human subject. The headgear may include an adjustment mechanism used for adjusting the headgear to conform with the human subject's head. These exemplary OPM modular assemblies and systems are described in more detail in U.S. Provisional Patent Application No. 63/170,892 filed Apr. 5, 2021, and U.S. Non-Provisional application Ser. No. 17/338,429, filed Jun. 3, 2021, previously incorporated by reference.

At least some of the elements of the OPM modular assemblies, systems which can employ the OPM modular assemblies, and methods of making and using the OPM modular assemblies have been disclosed in U.S. Patent Application Publications Nos. 2020/0072916; 2020/0056263; 2020/0025844; 2020/0057116; 2019/0391213; 2020/0088811; 2020/0057115; 2020/0109481; 2020/0123416; 2020/0191883; 2020/0241094; 2020/0256929; 2020/0309873; 2020/0334559; 2020/0341081; 2020/0381128; 2020/0400763; 2021/0011094; 2021/0015385; 2021/0041512; 2021/0041513; 2021/0063510; and 2021/0139742, and U.S. Provisional Patent Application Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; 63/031,469; 63/052,327; 63/076,015; 63/076,880; 63/080,248; 63/135,364; 63/136,415; and 63/170,892, all of which are incorporated herein by reference in their entireties.

In some examples, one or more components of brain interface system 102, FIG. 1, (e.g., one or more computing devices) may be configured to be located off the head of the user.

In each of the different brain interface system implementations described herein, the brain measurement data may be based on the type of operations performed by the different brain interface system implementations. For example, if brain interface system 102 is implemented by an optical measurement system configured to perform optical-based brain data acquisition operations, the brain measurement data may be based on the optical-based brain data acquisition operations. As another example, if brain interface system 102 is implemented by a multimodal measurement system configured to perform optical-based brain data acquisition operations and electrical-based brain data acquisition operations, the brain measurement data may be based on the optical-based brain data acquisition operations and the electrical-based brain data acquisition operations. As another example, if brain interface system 102 is implemented by a magnetic field measurement system configured to perform magnetic field-based brain data acquisition operations, the brain measurement data may be based on the magnetic field-based brain data acquisition operations.

In some examples, computing device 104 may be configured to obtain wellness therapy data representative of one or more characteristics of a wellness therapy and use the wellness therapy data in combination with the brain measurement data to modify an attribute of the wellness therapy. The wellness therapy may include any suitable treatment configured to treat a condition of a user. For instance, wellness therapy may include a stimulation therapy provided by a stimulation device, a stimulation pain management therapy provided by a stimulation device, a pharmaceutical therapy (e.g., a prescription and/or over-the-counter medication, a topical treatment, an oral treatment, etc.), or any other suitable therapy configured to treat a condition. A condition may include any state of health of a user that the user may wish to change, such as a disease, a symptom of a disease, a symptom of an injury, a disorder, a physical state, a mental state, an emotional state, etc. Example conditions may include pain, headache, sciatica, arthritis, inflammation, depression, epilepsy, stroke, autoimmune diseases, heart failure, lung failure, obesity, etc.

Computing device 104 may modify the attribute of the wellness therapy based on the brain measurement data in any suitable manner. For instance, computing device 104 may be configured to determine, based on the brain measurement data, a characteristic of the user and modify the attribute based on the characteristic. For example, the characteristic may include a perceived pain level of the user, a state of the condition being treated by the wellness therapy, a physical state of the user, a mental state of the user, etc.

Example mental states include, but are not limited to, joy, excitement, relaxation, surprise, fear, stress, anxiety, sadness, anger, disgust, contempt, contentment, calmness, approval, focus, attention, creativity, cognitive assessment, positive or negative reflections/attitude on experiences or the use of objects, etc. Further details on the methods and systems related to a predicted brain state, behavior, preferences, or attitude of the user, and the creation, training, and use of neuromes can be found in U.S. patent application Ser. No. 17/188,298, filed Mar. 1, 2021, issued as U.S. Pat. No. 11,132,625. Exemplary measurement systems and methods using biofeedback for awareness and modulation of mental state are described in more detail in U.S. patent application Ser. No. 16/364,338, filed Mar. 26, 2019, issued as U.S. Pat. No. 11,006,876. Exemplary measurement systems and methods used for detecting and modulating the mental state of a user using entertainment selections, e.g., music, film/video, are described in more detail in U.S. patent application Ser. No. 16/835,972, filed Mar. 31, 2020, issued as U.S. Pat. No. 11,006,878. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, issued as U.S. Pat. No. 11,172,869. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. patents and publications are incorporated herein by reference in their entirety.

In some examples, computing device 104 may obtain brain measurement data from brain interface system 102 prior to the user receiving the wellness therapy (or at any other time while the user is not actively receiving the wellness therapy). Such brain measurement data prior to the wellness therapy may provide a baseline brain measurement data with which computing device 104 may compare brain measurement data obtained concurrent to the wellness therapy. Computing device 104 may the modify the attribute of the wellness therapy based on a comparison of the brain measurement data obtained during wellness therapy to the baseline brain measurement data. For instance, the comparing of the brain measurement data during wellness therapy to the baseline brain measurement data may show an efficacy of the wellness therapy (e.g., a decrease in perceived pain) or an inefficacy of the wellness therapy and attributes of the wellness therapy may be modified accordingly.

In some examples, the brain measurement data obtained by computing device 104 during the wellness therapy may be analyzed based on time periods. For instance, the brain measurement data may be divided into a first set of brain measurement data and a second set of activity data, where each set corresponds to a first and a second time period, respectively, that the user receives the wellness therapy. Computing device 104 may compare the first and second set of brain measurement data and modify the attribute of the wellness therapy based on the comparing. Further, in some examples, computing device 104 may further modify the attribute of the wellness therapy between the first time period and the second time period (e.g., during a wellness therapy treatment session and/or in between wellness therapy treatment sessions). By modifying the attribute and comparing brain measurement data before and after the modifying, computing device 104 may determine an effect of the modifying of the attribute and further modify the attribute accordingly. This process may be repeated so that computing device 104 may be configured to continuously optimize the wellness therapy in a manner personalized to the user.

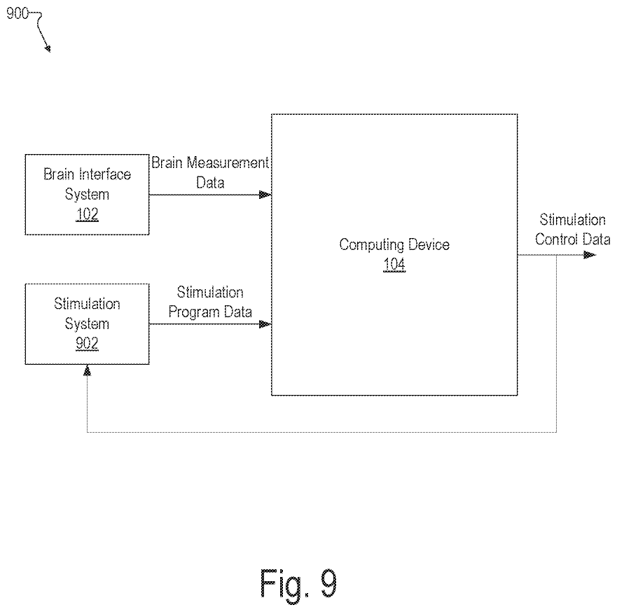

To illustrate, FIG. 9 shows an exemplary configuration 900 in which a stimulation system 902 presents a wellness therapy to a user. The wellness therapy may include a stimulation therapy provided by stimulation system 902. Stimulation system 902 may include any suitable device or devices configured to present electrical, magnetic, and/or mechanical stimulation to a user. For example, stimulation system 902 may include a stimulation device such as a non-invasive vagus nerve stimulation (nVNS) device, a transdermal electrical stimulation (TES) device, a transcranial magnetic stimulation (TMS) device, a transcranial electrical stimulation (tES) device (e.g., a transcranial direct current stimulation (tDCS) device and/or an transcranial alternating current stimulation (tACS) device), an implantable stimulation device, an implantable spinal cord stimulation device, etc. Additionally or alternatively, stimulation system 902 may include any other suitable stimulation device.

As shown, computing device 104 may obtain, from stimulation system 902, stimulation program data representative of a stimulation program. For example, computing device 104 may access the stimulation program data in substantially real time as stimulation system 902 applies the stimulation program to the user. Additionally or alternatively, the stimulation program data may be accessed prior to the stimulation program being applied to the user. The stimulation program may include parameters defining the stimulation therapy prescribed for the user. For instance, parameters may include a magnitude of the stimulation therapy, a frequency of the stimulation therapy, a pattern of the stimulation therapy, a stimulation site to which the stimulation therapy is applied, etc. In some examples, stimulation system 902 may include one or more preset stimulation programs and/or parameters that may be used as an initial stimulation therapy for the user. Additionally or alternatively, computing device 104 may provide (e.g., transmit) a stimulation program to stimulation system 902.

Computing device 104 may further obtain brain measurement data from brain interface system 102. For example, the brain measurement data may be obtained in substantially real time while stimulation system 902 is applying the stimulation program to the user. Computing device 104 may modify, based on the brain measurement data, an attribute of the stimulation therapy (e.g., a parameter of the stimulation program). For instance, computing device 104 may output stimulation control data that may be fed back into stimulation system 902 to modify one or more attributes of the stimulation therapy being applied and/or to be applied to the user by stimulation system 902. Computing device 104 may modify the attribute of the stimulation therapy in any suitable manner as described herein.

In some examples, computing device 104 may be further configured to transmit data and/or commands to control brain interface system 102. For instance, computing device 104 may transmit data and/or commands to brain interface system 102 that cause brain interface system 102 to acquire brain activity measurements of the user, transmit brain measurement data representative of the brain activity measurements, etc.

In some examples, computing device 104 may be included in stimulation system 902. In other examples, stimulation system 902 may be included in computing device 104. In other examples, computing device 104 and stimulation system 902 may be included in brain interface system 102.

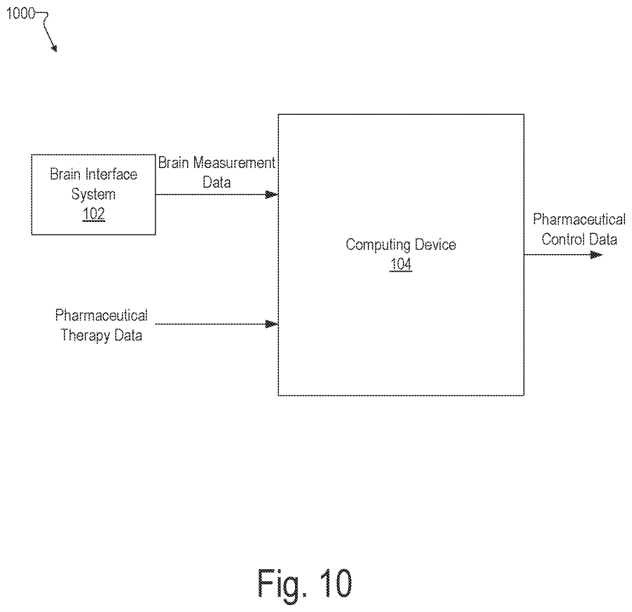

FIG. 10 shows an exemplary configuration 1000 in which a wellness therapy includes pharmaceutical therapy provided to a user. Computing device 104 may obtain pharmaceutical therapy data representative of the pharmaceutical therapy received by the user. For instance, the pharmaceutical therapy may include a pharmaceutical treatment of one or more particular pharmaceuticals at a particular dosage and frequency.

Computing device 104 may obtain brain measurement data and modify, based on the brain measurement data, an attribute of the pharmaceutical therapy (e.g., a dosage, a frequency, and/or a type of pharmaceutical) applied to the user. Computing device 104 may modify the attribute of the pharmaceutical therapy in any suitable manner as described herein.

Computing device 104 may output pharmaceutical control data representative of the modification. For instance, computing device 104 may output an indication of the modification and/or a modified pharmaceutical therapy treatment to the user. In some examples, a providing of the pharmaceutical therapy may involve another computing device (e.g., a computing device that dispenses a particular dosage of a pharmaceutical at a particular frequency) that may operate based on the pharmaceutical therapy data. In such examples, computing device 104 may be configured to obtain pharmaceutical therapy data from the other computing device and transmit pharmaceutical control data to the other computing device.

In some examples, computing device 104 may be configured to obtain sensor data associated with the user from a sensor not included in the brain interface system and further base the modification of the attribute of the wellness therapy on the sensor data.

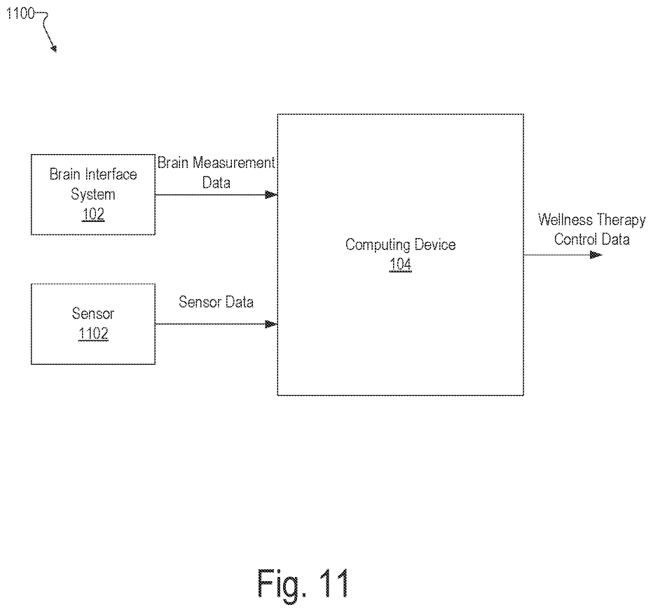

To illustrate, FIG. 11 shows an exemplary configuration 1100 in which a sensor 1102 outputs sensor data associated with the user. As shown, computing device 104 may obtain the sensor data (e.g., by receiving or otherwise accessing the sensor data in substantially real time as the sensor data is output by sensor 1102). In the example of FIG. 11, the wellness therapy control data output by computing device 104 is based on both the brain measurement data and the sensor data.

Sensor 1102 may be implemented in any suitable manner. For example, sensor 1102 may be implemented by one or more sensors that perform eye tracking, electrodermal activity (EDA)/conductance, pupillometry, heart rate, heart rate variability, and/or pulse oximetry. Additionally or alternatively, sensor 1102 may be implemented by one or more microphones configured to detect ambient sound of the user while the user receives the wellness therapy, one or more inertial motion units (IMUs) configured to detect movement by the user while the user receives the wellness therapy, etc. Output from one or more of these sensors may be fused to provide a wholistic view of the user's experience or state while receiving the wellness therapy. An example of this type of sensor is described in U.S. patent application Ser. No. 17/550,387, filed Dec. 14, 2021 and incorporated herein by reference in its entirety.

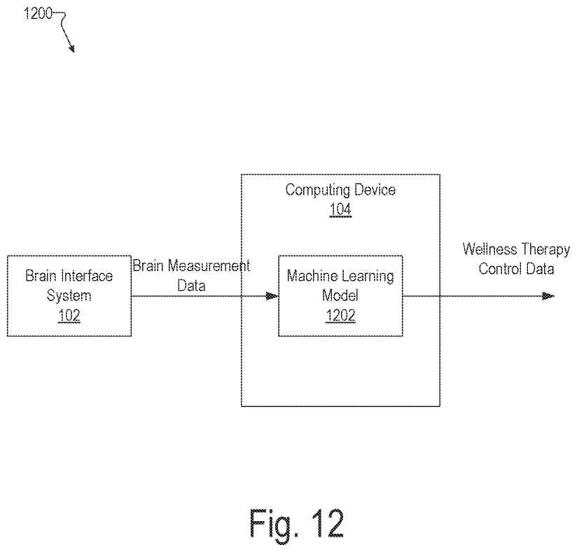

FIG. 12 shows an illustrative configuration 1200 in which computing device 104 is configured to implement a machine learning model 1202 to perform one or more operations based on the brain measurement data output by brain interface system 102. Such operations may include any of the operations described herein (e.g., modifying an attribute of a wellness therapy, etc.). As another example, computing device 104 may be configured to use machine learning model 1202 to determine which attribute and/or how to modify the attribute of the wellness therapy based on brain measurement data. For instance, machine learning model 1202 may be configured to determine correlations between particular attributes and efficacy of the wellness therapy based on comparisons of brain measurement data.

Machine learning model 1202 may be configured to perform any suitable machine learning heuristic (also referred to as artificial intelligence heuristic) to input data, which may be in either the time or frequency domains. Machine learning model 1202 may accordingly be supervised and/or unsupervised as may serve a particular implementation and may be configured to implement one or more decision tree learning algorithms, association rule learning algorithms, artificial neural network learning algorithms, deep learning algorithms, bitmap algorithms, and/or any other suitable data analysis technique as may serve a particular implementation.

In some examples, machine learning model 1202 is implemented by one or more neural networks, such as one or more deep convolutional neural networks (CNN) using internal memories of its respective kernels (filters), recurrent neural networks (RNN), and/or long/short term memory neural networks (LSTM). Machine learning model 1202 may be multi-layer. For example, machine learning model 1202 may be implemented by a neural network that includes an input layer, one or more hidden layers, and an output layer. Machine learning model 1202 may be trained in any suitable manner.

In some examples, computing device 104 may be configured to present, by way of a graphical user interface, content representative of the brain measurement data to the user. For instance, computing device 104 may record (e.g., store) wellness therapy data and corresponding brain measurement data and/or characteristics of the user based on the brain measurement data. Computing device 104 may present the recorded therapy history data to the user using metrics, graphics, etc. as described in U.S. patent application Ser. No. 17/559,316, filed Dec. 22, 2021, and incorporated herein by reference in its entirety.

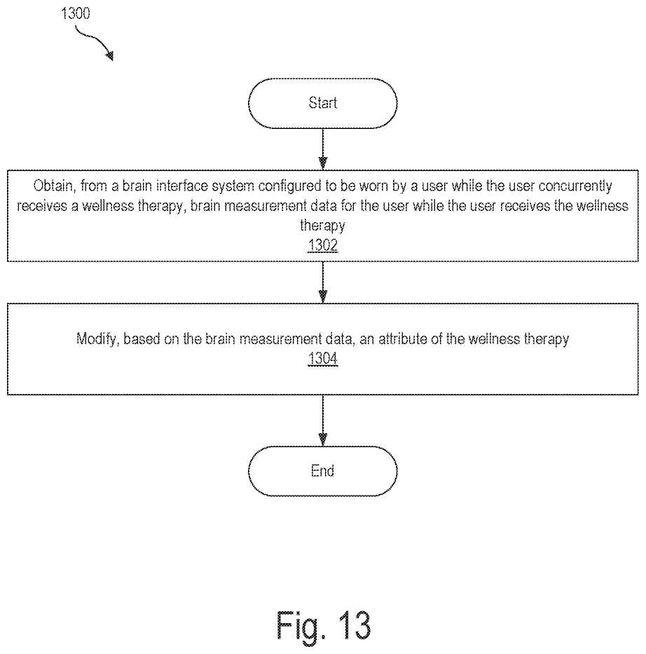

FIG. 13 illustrates an exemplary method 1300. While FIG. 13 illustrates exemplary operations according to one embodiment, other embodiments may omit, add to, reorder, and/or modify any of the operations shown in FIG. 13. One or more of the operations shown in FIG. 13 may be performed by computing device 104 and/or any implementation thereof. Each of the operations illustrated in FIG. 13 may be performed in any suitable manner.

At operation 1302, a computing device may obtain, from a brain interface system configured to be worn by a user while the user concurrently receives a wellness therapy, brain measurement data for the user while the user receives the wellness therapy.

At operation 1304, the computing device may modify, based on the brain measurement data, an attribute of the wellness therapy.

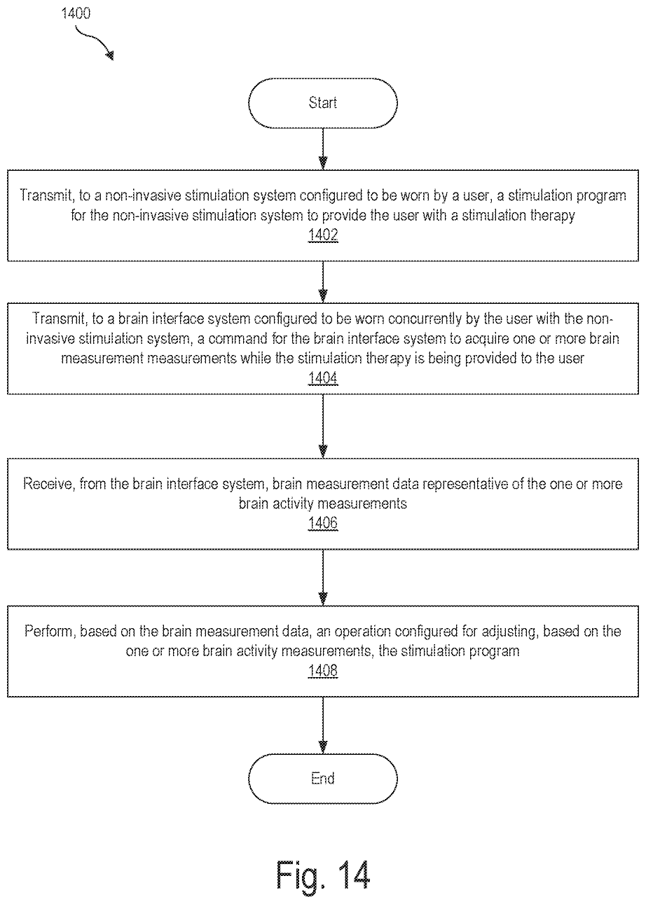

FIG. 14 illustrates an exemplary method 1400. While FIG. 14 illustrates exemplary operations according to one embodiment, other embodiments may omit, add to, reorder, and/or modify any of the operations shown in FIG. 14. One or more of the operations shown in FIG. 14 may be performed by computing device 104 and/or any implementation thereof. Each of the operations illustrated in FIG. 14 may be performed in any suitable manner.

At operation 1402, a computing device may transmit, to a non-invasive stimulation system configured to be worn by a user, a stimulation program for the non-invasive stimulation system to provide the user with a stimulation therapy.

At operation 1404, the computing device may transmit, to a brain interface system configured to be worn concurrently by the user with the non-invasive stimulation system, a command for the brain interface system to acquire one or more brain activity measurements while the stimulation therapy is being provided to the user.

At operation 1406, the computing device may receive, from the brain interface system, brain measurement data representative of the one or more brain activity measurements.

At operation 1408, the computing device may perform, based on the brain measurement data, an operation configured for modifying, based on the one or more brain activity measurements, the stimulation program.

Alternatively, the method shown in FIG. 14, may also apply to other known stimulation devices, e.g., a non-invasive vagus nerve stimulation (nVNS) device, a transdermal electrical stimulation (TES) device, a transcranial magnetic stimulation (TMS) device, a transcranial electrical stimulation (tES) device (e.g., a transcranial direct current stimulation (tDCS) device and/or an transcranial alternating current stimulation (tACS) device), an implantable stimulation device, an implantable spinal cord stimulation device, etc.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

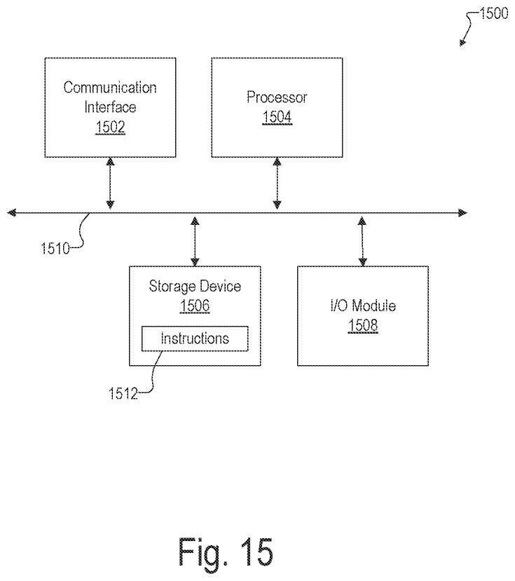

FIG. 15 illustrates an exemplary computing device 1500 that may be specifically configured to perform one or more of the processes described herein. Any of the systems, units, computing devices, and/or other components described herein may be implemented by computing device 1500.

As shown in FIG. 15, computing device 1500 may include a communication interface 1502, a processor 1504, a storage device 1506, and an input/output (“VO”) module 1508 communicatively connected one to another via a communication infrastructure 1510. While an exemplary computing device 1500 is shown in FIG. 15, the components illustrated in FIG. 15 are not intended to be limiting. Additional or alternative components may be used in other embodiments. Components of computing device 1500 shown in FIG. 15 will now be described in additional detail.

Communication interface 1502 may be configured to communicate with one or more computing devices. Examples of communication interface 1502 include, without limitation, a wired network interface (such as a network interface card), a wireless network interface (such as a wireless network interface card), a modem, an audio/video connection, and any other suitable interface.

Processor 1504 generally represents any type or form of processing unit capable of processing data and/or interpreting, executing, and/or directing execution of one or more of the instructions, processes, and/or operations described herein. Processor 1504 may perform operations by executing computer-executable instructions 1512 (e.g., an application, software, code, and/or other executable data instance) stored in storage device 1506.

Storage device 1506 may include one or more data storage media, devices, or configurations and may employ any type, form, and combination of data storage media and/or device. For example, storage device 1506 may include, but is not limited to, any combination of the non-volatile media and/or volatile media described herein. Electronic data, including data described herein, may be temporarily and/or permanently stored in storage device 1506. For example, data representative of computer-executable instructions 1512 configured to direct processor 1504 to perform any of the operations described herein may be stored within storage device 1506. In some examples, data may be arranged in one or more databases residing within storage device 1506.

I/O module 1508 may include one or more VO modules configured to receive user input and provide user output. I/O module 1508 may include any hardware, firmware, software, or combination thereof supportive of input and output capabilities. For example, I/O module 1508 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1508 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1508 is configured to provide graphical data to a display for presentation to a user. The graphical data may be representative of one or more graphical user interfaces and/or any other graphical content as may serve a particular implementation.

An illustrative system includes a brain interface system configured to be worn by a user and to output brain measurement data representative of brain activity of the user while the user concurrently receives a wellness therapy and a computing device configured to obtain the brain measurement data, and modify, based on the brain measurement data, an attribute of the wellness therapy.

An illustrative system includes a memory storing instructions and a processor communicatively coupled to the memory and configured to execute the instructions to obtain, from a brain interface system configured to be worn by a user while the user concurrently receives a wellness therapy, brain measurement data representative of brain activity of the user while the user receives the wellness therapy; and modify, based on the brain measurement data, an attribute of the wellness therapy.

An illustrative method includes obtaining, by a computing device from a brain interface system configured to be worn by a user while the user concurrently receives a wellness therapy, brain measurement data representative of brain activity of the user while the user receives the wellness therapy; and modifying, by the computing device and based on the brain measurement data, an attribute of the wellness therapy.

An illustrative non-transitory computer-readable medium storing instructions that, when executed, direct a processor of a computing device to obtain, from a brain interface system configured to be worn by a user while the user concurrently receives a wellness therapy, brain measurement data representative of brain activity of the user while the user receives the wellness therapy; and modify, based on the brain measurement data, an attribute of the wellness therapy.

An illustrative system includes a non-invasive stimulation system configured to be worn by a user, and provide the user with a wellness therapy; a brain interface system configured to be worn by the user and to output brain measurement data representative of brain activity of the user while the user concurrently receives the wellness therapy; and a computing device configured to obtain, from the brain interface system, the brain measurement data, and output, to the non-invasive stimulation system and based on the brain measurement data, a command to modify an attribute of the wellness therapy.

An illustrative system includes a brain interface system configured to be worn by a user and to output brain measurement data representative of brain activity of the user while the user concurrently receives a wellness therapy and a computing device configured to transmit, to a non-invasive stimulation system configured to be worn by a user, a stimulation program for the non-invasive stimulation system to provide the user with a stimulation therapy; transmit, to a brain interface system configured to be worn concurrently by the user with the non-invasive stimulation system, a command for the brain interface system to acquire one or more brain activity measurements while the stimulation therapy is being provided to the user; receive, from the brain interface system, brain measurement data representative of the one or more brain activity measurements; and perform, based on the brain measurement data, an operation configured for adjusting, based on the one or more brain activity measurements, the stimulation program.

An illustrative system includes a memory storing instructions and a processor communicatively coupled to the memory and configured to execute the instructions to transmit, to a non-invasive stimulation system configured to be worn by a user, a stimulation program for the non-invasive stimulation system to provide the user with a stimulation therapy; transmit, to a brain interface system configured to be worn concurrently by the user with the non-invasive stimulation system, a command for the brain interface system to acquire one or more brain activity measurements while the stimulation therapy is being provided to the user; receive, from the brain interface system, brain measurement data representative of the one or more brain activity measurements; and perform, based on the brain measurement data, an operation configured for adjusting, based on the one or more brain activity measurements, the stimulation program.

An illustrative method includes transmitting, by a computing device to a non-invasive stimulation system configured to be worn by a user, a stimulation program for the non-invasive stimulation system to provide the user with a stimulation therapy; transmitting, by the computing device to a brain interface system configured to be worn concurrently by the user with the non-invasive stimulation system, a command for the brain interface system to acquire one or more brain activity measurements while the stimulation therapy is being provided to the user; receiving, by the computing device from the brain interface system, brain measurement data representative of the one or more brain activity measurements; and performing, by the computing device and based on the brain measurement data, an operation configured for adjusting, based on the one or more brain activity measurements, the stimulation program.

An illustrative non-transitory computer-readable medium storing instructions that, when executed, direct a processor of a computing device to transmit, to a non-invasive stimulation system configured to be worn by a user, a stimulation program for the non-invasive stimulation system to provide the user with a stimulation therapy; transmit, to a brain interface system configured to be worn concurrently by the user with the non-invasive stimulation system, a command for the brain interface system to acquire one or more brain activity measurements while the stimulation therapy is being provided to the user; receive, from the brain interface system, brain measurement data representative of the one or more brain activity measurements; and perform, based on the brain measurement data, an operation configured for adjusting, based on the one or more brain activity measurements, the stimulation program.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a brain interface system configured to be worn by a user and to output brain measurement data representative of brain activity of the user while the user concurrently receives a wellness therapy; and
a computing device configured to:
obtain the brain measurement data, and
modify, based on the brain measurement data, an attribute of the wellness therapy.

2. The system of claim 1, wherein the brain interface system comprises an optical measurement system configured to perform optical-based brain data acquisition operations, the brain measurement data based on the optical-based brain data acquisition operations.

3. The system of claim 2, wherein the optical measurement system comprises:
a wearable assembly configured to be worn by the user and comprising:
a plurality of light sources each configured to emit light directed at a brain of the user, and
a plurality of detectors configured to detect arrival times for photons of the light after the light is scattered by the brain, the brain measurement data based on the arrival times.

4. The system of claim 3, wherein the detectors each comprise a plurality of single-photon avalanche diode (SPAD) circuits.

5. The system of claim 3, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

6. The system of claim 5, wherein the first and second modules are configured to be removably attached to the wearable assembly.

7. The system of claim 1, wherein the brain interface system comprises a multimodal measurement system configured to perform optical-based brain data acquisition operations and electrical-based brain data acquisition operations, the brain measurement data based on the optical-based brain data acquisition operations and the electrical-based brain data acquisition operations.

8. The system of claim 7, wherein the multimodal measurement system comprises:
a wearable assembly configured to be worn by the user and comprising:
a plurality of light sources each configured to emit light directed at a brain of the user,
a plurality of detectors configured to detect arrival times for photons of the light after the light is scattered by the brain, and
a plurality of electrodes configured to be external to the user and detect electrical activity of the brain, the brain activity based on the arrival times and the electrical activity.

9. The system of claim 8, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

10. The system of claim 9, wherein the plurality of electrodes comprises a first electrode on a surface of the first module and a second electrode on a surface of the second module.

11. The system of claim 10, wherein the first electrode surrounds the first light source on the surface of the first module.

12. The system of claim 1, wherein the brain interface system comprises a magnetic field measurement system configured to perform magnetic field-based brain data acquisition operations, the brain measurement data based on the magnetic field-based brain data acquisition operations.

13. The system of claim 12, wherein the magnetic field measurement system comprises a wearable sensor unit configured to be worn by the user and comprising a magnetometer configured to detect a magnetic field generated within a brain of the user.

14. The system of claim 1, wherein the wellness therapy comprises a stimulation therapy provided by a stimulation device, the stimulation therapy configured to treat a condition of the user.

15. The system of claim 14, wherein the stimulation device comprises one or more of a non-invasive stimulation system configured to be worn by the user or a stimulation device configured to be implanted within the user.

16. The system of claim 15, wherein the non-invasive stimulation system comprises one or more of a non-invasive vagus nerve stimulation (nVNS) device, a transdermal electrical stimulation (TES) device, a transcranial magnetic stimulation (TMS) device, or a transcranial electrical stimulation (tES) device.

17. The system of claim 14, wherein the attribute of the wellness therapy comprises a parameter associated with the stimulation therapy.

18. The system of claim 17, wherein the parameter one or more of a magnitude of the stimulation therapy, a frequency of the stimulation therapy, a pattern of the stimulation therapy, a program that controls the stimulation therapy, or a stimulation site to which the stimulation therapy is applied.

19. The system of claim 1, wherein the wellness therapy comprises a pharmaceutical treatment configured to treat a condition of the user.

20. The system of claim 19, wherein the attribute of the wellness therapy comprises one or more of a dosage of the pharmaceutical treatment, a frequency at which the pharmaceutical treatment, or a type of pharmaceutical for the pharmaceutical treatment is applied to the user.

21. The system of claim 1, wherein:
the computing device is further configured to:
obtain baseline brain measurement data representative of brain activity of the user prior to the user receiving the wellness therapy, and
compare the brain measurement data to the baseline brain measurement data; and
the modifying the attribute of the wellness therapy is further based on the comparing the brain measurement data to the baseline brain measurement data.

22. The system of claim 1, wherein:
the brain measurement data comprises a first set of brain measurement data representative of brain activity of the user during a first time period while the user concurrently receives the wellness therapy and a second set of brain measurement data representative of brain activity of the user during a second time period while the user concurrently receives a wellness therapy;
the computing device is further configured to compare the first set of brain measurement data with the second set of brain measurement data; and
the modifying the attribute of the wellness therapy is further based on the comparing of the first set of brain measurement data with the second set of brain measurement data.

23. The system of claim 22, wherein the computing device is further configured to modify the attribute of the wellness therapy between the first time period and the second time period.

24. A system comprising:
a memory storing instructions; and
a processor communicatively coupled to the memory and configured to execute the instructions to:
obtain, from a brain interface system configured to be worn by a user while the user concurrently receives a wellness therapy, brain measurement data representative of brain activity of the user while the user receives the wellness therapy; and
modify, based on the brain measurement data, an attribute of the wellness therapy.

25. The system of claim 24, wherein the wellness therapy comprises a stimulation therapy provided by a stimulation device, the stimulation therapy configured to treat a condition of the user.

26. The system of claim 25, wherein the stimulation device comprises one or more of a non-invasive stimulation system configured to be worn by the user or a stimulation device configured to be implanted within the user.

27. The system of claim 26, wherein the non-invasive stimulation system comprises one or more of a non-invasive vagus nerve stimulation (nVNS) device, a transdermal electrical stimulation (TES) device, a transcranial magnetic stimulation (TMS) device, or a transcranial electrical stimulation (tES) device.

28. The system of claim 25, wherein the attribute of the wellness therapy comprises a parameter associated with the stimulation therapy.

29. The system of claim 28, wherein the parameter one or more of a magnitude of the stimulation therapy, a frequency of the stimulation therapy, a pattern of the stimulation therapy, a program that controls the stimulation therapy, or a stimulation site to which the stimulation therapy is applied.

30. The system of claim 24, wherein the wellness therapy comprises a pharmaceutical treatment configured to treat a condition of the user.

31. The system of claim 30, wherein the attribute of the wellness therapy comprises one or more of a dosage of the pharmaceutical treatment, a frequency at which the pharmaceutical treatment, or a type of pharmaceutical for the pharmaceutical treatment is applied to the user.

32. The system of claim 24, wherein:
the processor further configured to execute the instructions to:
obtain baseline brain measurement data representative of brain activity of the user prior to the user receiving the wellness therapy, and
compare the brain measurement data to the baseline brain measurement data; and
the modifying the attribute of the wellness therapy is further based on the comparing the brain measurement data to the baseline brain measurement data.

33. The system of claim 24, wherein:
the brain measurement data comprises a first set of brain measurement data representative of brain activity of the user during a first time period while the user concurrently receives the wellness therapy and a second set of brain measurement data representative of brain activity of the user during a second time period while the user concurrently receives a wellness therapy;
the processor further configured to execute the instructions to compare the first set of brain measurement data with the second set of brain measurement data; and
the modifying the attribute of the wellness therapy is further based on the comparing of the first set of brain measurement data with the second set of brain measurement data.

34. The system of claim 33, wherein the processor further configured to execute the instructions to modify the attribute of the wellness therapy between the first time period and the second time period.

35. A method comprising:
obtaining, by a computing device from a brain interface system configured to be worn by a user while the user concurrently receives a wellness therapy, brain measurement data representative of brain activity of the user while the user receives the wellness therapy; and
modifying, by the computing device and based on the brain measurement data, an attribute of the wellness therapy.

36. A non-transitory computer-readable medium storing instructions that, when executed, direct a processor of a computing device to:
obtain, from a brain interface system configured to be worn by a user while the user concurrently receives a wellness therapy, brain measurement data representative of brain activity of the user while the user receives the wellness therapy; and
modify, based on the brain measurement data, an attribute of the wellness therapy.

37. A system comprising:
a non-invasive stimulation system configured to:
be worn by a user, and
provide the user with a wellness therapy;
a brain interface system configured to be worn by the user and to output brain measurement data representative of brain activity of the user while the user concurrently receives the wellness therapy; and
a computing device configured to:
obtain, from the brain interface system, the brain measurement data, and
output, to the non-invasive stimulation system and based on the brain measurement data, a command to modify an attribute of the wellness therapy.

38. The system of claim 37, wherein the brain interface system comprises an optical measurement system configured to perform optical-based brain data acquisition operations, the brain measurement data based on the optical-based brain data acquisition operations.

39. The system of claim 38, wherein the optical measurement system comprises:
a wearable assembly configured to be worn by the user and comprising:
a plurality of light sources each configured to emit light directed at a brain of the user, and
a plurality of detectors configured to detect arrival times for photons of the light after the light is scattered by the brain, the brain measurement data based on the arrival times.

40. The system of claim 39, wherein the detectors each comprise a plurality of single-photon avalanche diode (SPAD) circuits.

41. The system of claim 39, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

42. The system of claim 41, wherein the first and second modules are configured to be removably attached to the wearable assembly.

43. The system of claim 37, wherein the brain interface system comprises a magnetic field measurement system configured to perform magnetic field-based brain data acquisition operations, the brain measurement data based on the magnetic field-based brain data acquisition operations.

44. The system of claim 43, wherein the magnetic field measurement system comprises a wearable sensor unit configured to be worn by the user and comprising a magnetometer configured to detect a magnetic field generated within a brain of the user.

45. The system of claim 37, wherein the brain interface system comprises a multimodal measurement system configured to perform optical-based brain data acquisition operations and electrical-based brain data acquisition operations, the brain measurement data based on the optical-based brain data acquisition operations and the electrical-based brain data acquisition operations.

46. The system of claim 45, wherein the multimodal measurement system comprises:
a wearable assembly configured to be worn by the user and comprising:
a plurality of light sources each configured to emit light directed at a brain of the user,
a plurality of detectors configured to detect arrival times for photons of the light after the light is scattered by the brain, and
a plurality of electrodes configured to be external to the user and detect electrical activity of the brain, the brain activity based on the arrival times and the electrical activity.

47. The system of claim 46, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

48. The system of claim 47, wherein the plurality of electrodes comprises a first electrode on a surface of the first module and a second electrode on a surface of the second module.

49. The system of claim 48, wherein the first electrode surrounds the first light source on the surface of the first module.

50. The system of claim 37, wherein the wellness therapy comprises a stimulation therapy provided by the non-invasive stimulation system, the stimulation therapy configured to treat a condition of the user.

51. The system of claim 50, wherein the attribute of the wellness therapy comprises a parameter associated with the stimulation therapy.

52. The system of claim 51, wherein the parameter one or more of a magnitude of the stimulation therapy, a frequency of the stimulation therapy, a pattern of the stimulation therapy, a program that controls the stimulation therapy, or a stimulation site to which the stimulation therapy is applied.

53. The system of claim 37, wherein the non-invasive stimulation system comprises one or more of a non-invasive vagus nerve stimulation (nVNS) device, a transdermal electrical stimulation (TES) device, a transcranial magnetic stimulation (TMS) device, or a transcranial electrical stimulation (tES) device.

54. The system of claim 37, wherein:
the computing device is further configured to:
obtain baseline brain measurement data representative of brain activity of the user prior to the user receiving the wellness therapy, and
compare the brain measurement data to the baseline brain measurement data; and
the modifying the attribute of the wellness therapy is further based on the comparing the brain measurement data to the baseline brain measurement data.

55. The system of claim 37, wherein:
the brain measurement data comprises a first set of brain measurement data representative of brain activity of the user during a first time period while the user concurrently receives the wellness therapy and a second set of brain measurement data representative of brain activity of the user during a second time period while the user concurrently receives a wellness therapy;
the computing device is further configured to compare the first set of brain measurement data with the second set of brain measurement data; and
the modifying the attribute of the wellness therapy is further based on the comparing of the first set of brain measurement data with the second set of brain measurement data.

56. The system of claim 55, wherein the computing device is further configured to modify the attribute of the wellness therapy between the first time period and the second time period.

57. A system comprising:
a brain interface system configured to be worn by a user and to output brain measurement data representative of brain activity of the user while the user concurrently receives a wellness therapy; and
a computing device configured to:
transmit, to a non-invasive stimulation system configured to be worn by a user, a stimulation program for the non-invasive stimulation system to provide the user with a stimulation therapy;
transmit, to a brain interface system configured to be worn concurrently by the user with the non-invasive stimulation system, a command for the brain interface system to acquire one or more brain activity measurements while the stimulation therapy is being provided to the user;
receive, from the brain interface system, brain measurement data representative of the one or more brain activity measurements; and
perform, based on the brain measurement data, an operation configured for adjusting, based on the one or more brain activity measurements, the stimulation program.

58. The system of claim 57, wherein the non-invasive stimulation system comprises one or more of a non-invasive vagus nerve stimulation (nVNS) device, a transdermal electrical stimulation (TES) device, a transcranial magnetic stimulation (TMS) device, or a transcranial electrical stimulation (tES) device.

59. The system of claim 57, wherein the adjusting the stimulation program comprises adjusting or more of a magnitude of the stimulation therapy, a frequency of the stimulation therapy, a pattern of the stimulation therapy, a program that controls the stimulation therapy, or a stimulation site to which the stimulation therapy is applied.

60. The system of claim 57, wherein:
the computing device is further configured to:
obtain baseline brain measurement data representative of brain activity of the user prior to the user receiving the stimulation therapy, and
compare the brain measurement data to the baseline brain measurement data; and
the adjusting the stimulation program is further based on the comparing the brain measurement data to the baseline brain measurement data.

61. The system of claim 57, wherein:
the brain measurement data comprises a first set of brain measurement data representative of brain activity of the user during a first time period while the user concurrently receives the stimulation therapy and a second set of brain measurement data representative of brain activity of the user during a second time period while the user concurrently receives a stimulation therapy;
the computing device is further configured to compare the first set of brain measurement data with the second set of brain measurement data; and
the adjusting the stimulation program is further based on the comparing of the first set of brain measurement data with the second set of brain measurement data.

62. The system of claim 61, wherein the computing device is further configured to perform an additional operation configured for adjusting the stimulation therapy between the first time period and the second time period.

63. A system comprising:
a memory storing instructions; and
a processor communicatively coupled to the memory and configured to execute the instructions to:
transmit, to a non-invasive stimulation system configured to be worn by a user, a stimulation program for the non-invasive stimulation system to provide the user with a stimulation therapy;
transmit, to a brain interface system configured to be worn concurrently by the user with the non-invasive stimulation system, a command for the brain interface system to acquire one or more brain activity measurements while the stimulation therapy is being provided to the user;
receive, from the brain interface system, brain measurement data representative of the one or more brain activity measurements; and
perform, based on the brain measurement data, an operation configured for adjusting, based on the one or more brain activity measurements, the stimulation program.

64. A method comprising:
transmitting, by a computing device to a non-invasive stimulation system configured to be worn by a user, a stimulation program for the non-invasive stimulation system to provide the user with a stimulation therapy;
transmitting, by the computing device to a brain interface system configured to be worn concurrently by the user with the non-invasive stimulation system, a command for the brain interface system to acquire one or more brain activity measurements while the stimulation therapy is being provided to the user;
receiving, by the computing device from the brain interface system, brain measurement data representative of the one or more brain activity measurements; and
performing, by the computing device and based on the brain measurement data, an operation configured for adjusting, based on the one or more brain activity measurements, the stimulation program.

65. A non-transitory computer-readable medium storing instructions that, when executed, direct a processor of a computing device to:
transmit, to a non-invasive stimulation system configured to be worn by a user, a stimulation program for the non-invasive stimulation system to provide the user with a stimulation therapy;
transmit, to a brain interface system configured to be worn concurrently by the user with the non-invasive stimulation system, a command for the brain interface system to acquire one or more brain activity measurements while the stimulation therapy is being provided to the user;
receive, from the brain interface system, brain measurement data representative of the one or more brain activity measurements; and
perform, based on the brain measurement data, an operation configured for adjusting, based on the one or more brain activity measurements, the stimulation program.
